(19) United States
(12) Patent Application Publication
(10) Pub. No.: US 2024/0131010 A1
(43) Pub. Date: Apr. 25, 2024

(54) SUSTAINED RELEASE OSMOTIC-CONTROLLED PHARMACEUTICAL COMPOSITION AND PREPARATION METHOD THEREOF

(71) Applicant: Anxo Pharmaceutical Co., Ltd., Taipei City (TW)

(72) Inventors: Chun-You LIOU, Taipei City (TW); Tzu-Hsien CHAN, Taipei City (TW); Hua-Jing JHAN, Taipei City (TW); I-Hsiang LIU, Taipei City (TW); Tse-Hsien CHEN, Taipei City (TW); Chi-Heng JIAN, Taipei City (TW)

(21) Appl. No.: 18/487,648

(22) Filed: Oct. 15, 2023

Related U.S. Application Data
(60) Provisional application No. 63/418,029, filed on Oct. 20, 2022.

(51) Int. Cl. A61K 31/427; A61K 9/20; A61K 9/28

(52) U.S. Cl. CPC A61K 31/427; A61K 9/2009; A61K 9/2054; A61K 9/2059; A61K 9/2893

(57) ABSTRACT

In some embodiments of the present disclosure, a sustained release osmotic-controlled pharmaceutical composition is provided, including: a core and a semi-permeable membrane coated on the core. The core includes a drug compartment, in which the drug compartment includes a first active ingredient, a first polymer and a first osmogen, and the first active ingredient includes lurasidone, a pharmaceutical acceptable salt of the lurasidone or a combination thereof. The semi-permeable membrane includes a membrane body and at least one pore distributed in the membrane body.

### CROSS-REFERENCE TO RELATED APPLICATION

This application claims priority to U.S. Provisional Application Ser. No. 63/418,029, filed Oct. 20, 2022, which is herein incorporated by reference in its entirety.

### BACKGROUND

### Field of Invention

The present disclosure relates to a sustained release osmotic-controlled pharmaceutical composition. In particular, the present disclosure relates to a sustained release osmotic-controlled pharmaceutical composition comprising lurasidone, a pharmaceutical acceptable salt of the lurasidone or a combination thereof.

### Description of Related Art

Latuda® (active pharmaceutical ingredient (API): Lurasidone HCl) is a rapid release (immediate release) tablet that allows lurasidone to be quickly released to the circulation system and achieves rapid onset of effect. However, since lurasidone is released into the blood quickly, the lager fluctuation of the lurasidone plasma concentration occurs, leading to safety concerns.

Despite several kinds of sustained and controlled release formulations have been provided, there are still many problems.

For example, CN110693843A provides tablets that are manufactured by a hydrophilic gel matrix system. The mechanism of API release in the hydrophilic gel matrix system is controlled by hydrophilic polymer, which will form a gel when absorbing liquid. Therefore, API is diffused out of the tablet along with the erosion of gel over time, and the purpose of the sustained and controlled release is achieved. However, the API release of this formulation is relatively fast in the early stage, representing a first-order release curve, and the release rate is affected by API concentration. Along with the decrease of API concentration, the release rate decreases. That is, the effect of decreasing the fluctuation of the plasma concentration is limited in the hydrophilic gel matrix system. Also, the risk of API burst release is relatively high since the structure of the tablet is quickly collapsed caused by the churning of the gastrointestinal tract while digested.

CN107998105A provides a pellet capsule formulation formed by a core with API, an isolation coating layer, an extended release coating layer and a protective coating layer. Since the pellet capsule includes multiple layers, multiple burdensome manufacturing procedures are required. Furthermore, due to low flexibility of the procedures and high technical requirement, the deviation between lots may increasingly occur. That is, multiple limitations exist in the pellet capsule formulation.

Therefore, how to provide a sustained release pharmaceutical composition which avoids the burst release of lurasidone and can be easily manufactured with stable performance remains to be solved.

### SUMMARY

In one aspect of the present disclosure, a sustained release osmotic-controlled pharmaceutical composition is provided, comprising: a core and a semi-permeable membrane coated on the core. The core comprises a drug compartment, in which the drug compartment comprises a first active ingredient, a first polymer and a first osmogen, and the first active ingredient comprises lurasidone, a pharmaceutical acceptable salt of the lurasidone or a combination thereof. The semi-permeable membrane comprises a membrane body and at least one pore distributed in the membrane body.

In some embodiments, a weight percentage of the first active ingredient is from 2%-30% based on 100% by weight of the core.

In some embodiments, a weight percentage of the first polymer is from 5% to 80% based on 100% by weight of the core.

In some embodiments, the first polymer comprises poly(methyl methacrylate), microcrystalline cellulose, methylcellulose, hydroxyethyl cellulose, hydroxypropyl cellulose, poly(ethylene oxide), polyoxypropylene, polyvinylpyrrolidone, carbomer, sodium carboxymethyl starch, carboxymethyl cellulose, a sodium salt of the carboxymethyl cellulose, cross-linked sodium carboxymethyl cellulose, xanthan gum or a combination thereof.

In some embodiments, the first osmogen comprises water-soluble salt, a carbohydrate, a water-soluble amino acid or a combination thereof, and a weight percentage of the first osmogen is from 10% to 55% based on 100% by weight of the core.

In some embodiments, the first osmogen comprises magnesium chloride, magnesium sulfate, lithium chloride, sodium chloride, sodium sulfate, sodium phosphate, potassium chloride, potassium phosphate, sodium acetate, potassium acetate, magnesium succinate, sodium benzoate, sodium citrate, sodium ascorbate, sodium carboxymethyl cellulose, sucrose, sorbitol, mannitol, glucose, lactose, fructose, glycine, leucine, alanine, methionine, urea or a combination thereof.

In some embodiments, the drug compartment further comprises an acidifier.

In some embodiments, a weight percentage of the semi-permeable membrane is from 1% to 45% based on 100% by weight of the core.

In some embodiments, the membrane body comprises cellulose acetate, ethyl cellulose or a combination thereof.

In some embodiments, the core further comprises a push compartment, and the push compartment comprises a second polymer and a second osmogen.

In some embodiments, a weight percentage of the second polymer is from 15% to 55% based on 100% by weight of the core, and a weight percentage of the second osmogen is from 5% to 55% based on 100% by weight of the core.

In some embodiments, when assayed in a Mcilvaine buffer in pH 3.8, a first dissolution percentage of the first active ingredient is from 0% to 30% within 2 hrs, and a second dissolution percentage of the first active ingredient is from 30% to 100% within 10 hrs.

In some embodiments, the sustained release osmotic-controlled pharmaceutical composition further comprises a film coating coated on the semi-permeable membrane.

In some embodiments, a weight percentage of the film coating is from 2% to 25% based on 100% by weight of the core and the semi-permeable membrane.

In some embodiments, the film coating comprises a second active ingredient and the second active ingredient comprises lurasidone, a pharmaceutical acceptable salt of the lurasidone or a combination thereof, in which a weight percentage of the second active ingredient is from 10% to 60% based on 100% by weight of the first active ingredient in the core.

In some embodiments, when assayed in a Mcilvaine buffer in pH 3.8, a dissolution percentage of the first active ingredient and the second active ingredient is from 10% to 40% within 1 hr.

In another aspect of the present disclosure, a method of preparing a sustained release osmotic-controlled pharmaceutical composition is provided, comprising: mixing a first active ingredient, a first polymer and a first osmogen to form a drug compartment mixture, in which the first active ingredient comprises lurasidone, a pharmaceutical acceptable salt of the lurasidone or a combination thereof; compressing and tableting the drug compartment mixture to form a core; mixing a membrane body material and a porogen in a solvent to form a membrane liquid; spraying the membrane liquid on the core; and drying the membrane liquid to form a semi-permeable membrane coated on the core.

In some embodiments, the method further comprises mixing a second polymer and a second osmogen to form a push compartment mixture; and compressing and tableting the drug compartment mixture comprises: compressing the drug compartment mixture to form a drug compartment; mixing the push compartment mixture with the drug compartment to form a core mixture; and compressing and tableting the core mixture to form the core.

In some embodiments, the method further comprises: mixing a film coating material in an aqueous solution to form a film coating liquid; and spraying the film coating liquid on the semi-permeable membrane to form a film coating coated on the semi-permeable membrane.

In another aspect of the present disclosure, a method of treating a mental disease is provided, comprising administering the abovementioned sustained release osmotic-controlled pharmaceutical composition to a subject suffered from the mental disease.

### BRIEF DESCRIPTION OF THE DRAWINGS

In order to make the above-mentioned and other objects, features, advantages and embodiments of the present disclosure more clearly understood, descriptions of accompanying drawings are as follows:

FIG. 1 illustrates a flow chart of a method of preparing a sustained release osmotic-controlled pharmaceutical composition in some embodiments of the present disclosure.

FIG. 2A illustrates the release profiles of Example 1 to Example 9 and the commercial product, Latuda, in the dissolution assay in some embodiments of the present disclosure.

FIG. 2B illustrates blood concentrations of Latuda in beagle dogs while feeding with Example 6 or Latuda in some embodiments of the present disclosure.

### DETAILED DESCRIPTION

In order that the present disclosure is described in detail and completeness, implementation aspects and specific embodiments of the present disclosure with illustrative description are presented, but those are not the only form for implementation or use of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein may include a plurality of sub-steps or actions.

### Definition

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, “drug” or “active ingredient” refers to lurasidone or its pharmaceutical acceptable salts, including but not limited to salts, esters, complexes, chelating agents, cage compounds, racemates, mirror image isomers, or the like.

As used herein, “water-soluble” refers to a substance that have a solubility in water of higher than 10 g/L (1 g/100 mL), such as from 10 g/L to 100 g/L, for example, 10 g/L, 20 g/L, 30 g/L, 40 g/L, 50 g/L, 60 g/L, 70 g/L, 80 g/L, 90 g/L, 100 g/L, or any value between any interval of the abovementioned values.

As used herein, “Cmax” refers to the maximum blood plasma/media concentration of the active ingredient.

As used herein, “zero-order release” is characterized by the fact that the release rate of the active ingredient is not affected by the concentration of the active ingredient, and the active ingredient is generally released at a constant or near constant rate. The zero-order release is represented as a straight line segment in the dissolution curve, in which the difference of the release rate at each point of the straight line segment and the average rate of the straight line segment is lower than 1%, and the interval of the two time points for sampling shall not exceed 2 hours.

As used herein, “excipients” refers to pharmaceutical additives without pharmacological activity and used in pharmaceutical compositions according to different purposes and functions.

As used herein, “osmogen” refers to the material that provides an osmotic pressure difference that drives the liquid in the environment into the sustained release osmotic-controlled pharmaceutical composition.

As used herein, “pore” refers to the opening formed by the porogen that allows the liquid in the environment into the sustained release osmotic-controlled pharmaceutical composition.

As used herein, “orifice” refers to the opening formed by artificial work, laser, mechanical method or other suitable orifice-making method for allowing the active ingredient push out of the sustained release osmotic-controlled pharmaceutical composition.

As used herein, “immediate release” (IR) refers to the phenomenon that the active ingredient is completely released in 2 hrs, 1 hr, 30 mins or less.

Sustained Release Osmotic-Controlled Pharmaceutical Composition and Preparation Method Thereof

The main purpose of the present disclosure is to provide a sustained release osmotic-controlled pharmaceutical composition with a sustained and controlled release osmotic pharmaceutical dosage form that controls the release of lurasidone or its pharmaceutically acceptable salt in an osmotic pressure manner. It's noted that lurasidone is insoluble in water (such as the solubility in water is lower than 0.1 mg/mL) and has high dose requirement in the dose regimen. Therefore, the intrinsic hurdle for applying the lurasidone in the sustained release osmotic-controlled pharmaceutical composition is related to how to increase the dissolution efficiency of the active ingredient in a steady release state but avoid fluctuation.

Surprisingly, the sustained release osmotic-controlled pharmaceutical composition achieves the sustained and controlled release through the application of osmogens and polymers in the core (such as drug compartment, or the combination of the drug compartment and the push compartment) and the semi-permeable membrane. The mechanism of the sustained release osmotic-controlled pharmaceutical composition is that when the liquid in the environment diffuses into the core by the osmotic pressure caused by the osmogens, the polymer swells to form a three-dimensional structure, and the active ingredient dispersed in the polymer is dissolved by the liquid in the environment and released slowly through diffusion. Meanwhile, the three-dimensional structure formed by the polymer can act as a barrier for delaying the diffusion of the active ingredient. On the other side, the semi-permeable membrane serves as a barrier that allows the liquid in the environment to pass and to control the release of the active ingredient.

It should be emphasized that the active ingredient in the sustained release osmotic-controlled pharmaceutical composition is released at a zero-order rate for at least 4 hrs, not affected by the environment, through the osmotic-controlled mechanism, and the plateau of the dissolution percentage of the active ingredient is higher than 50%. Therefore, the sustained release osmotic-controlled pharmaceutical composition of the present disclosure can not only stably provide desired amount of the active ingredient but also significantly reduce Cmax and avoid the fluctuation of the active ingredient, prolonging the release of the active ingredient (such as, higher than 4 hr), thereby reducing the occurrence of side effects, and increasing the patient's compliance with medication.

That is, the sustained release osmotic-controlled pharmaceutical composition of the present disclosure balances the requirements of the prevention of the fluctuation of active ingredient plasma concentration and the higher release efficiency of the active ingredient by the formulation design and selection of the appropriate materials.

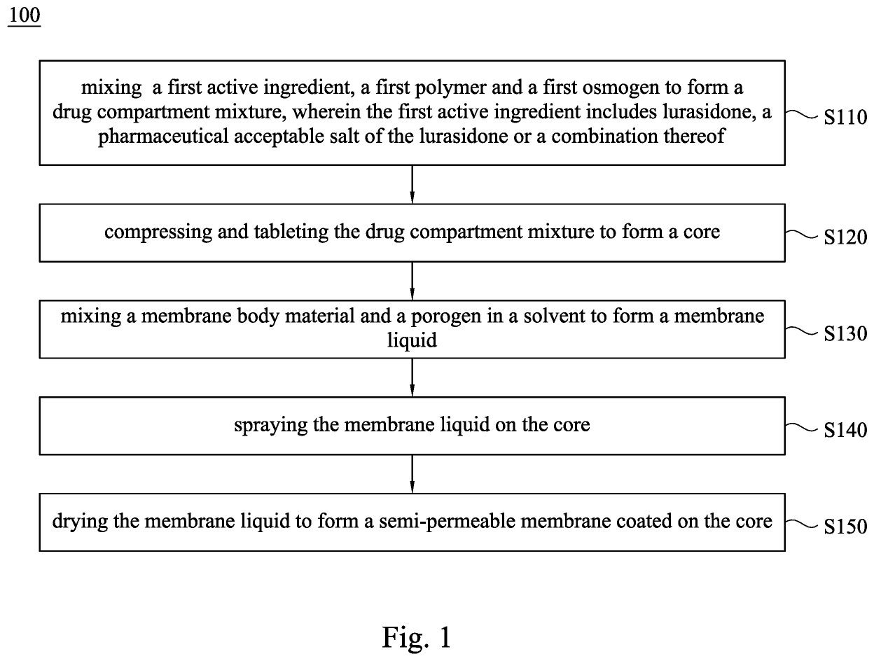

Please refer to FIG. 1, representing a flow chart of a method 100 of preparing a sustained release osmotic-controlled pharmaceutical composition in some embodiments of the present disclosure, comprising step S110, step S120, step S130, step S140 and step S150. It should be emphasized that compared with the capsule formulation described in CN107998105A, the method 100 is easier and the quantity between lots can be relatively stable.

First of all, refer to step S110, mixing a first active ingredient, a first polymer and a first osmogen to form a drug compartment mixture, in which the first active ingredient comprises lurasidone, a pharmaceutical acceptable salt of the lurasidone or a combination thereof.

In some embodiments, the first polymer comprises poly(methyl methacrylate), microcrystalline cellulose, methylcellulose, hydroxyethyl cellulose, hydroxypropyl cellulose, poly(ethylene oxide), polyoxypropylene, polyvinylpyrrolidone, carbomer, sodium carboxymethyl starch, carboxymethyl cellulose, a sodium salt of the carboxymethyl cellulose, cross-linked sodium carboxymethyl cellulose, xanthan gum or a combination thereof. The first polymer expands while the liquid in the environment diffuses into the sustained release osmotic-controlled pharmaceutical composition and delays the release the first active ingredient.

In some embodiments, the first osmogen comprises a water-soluble salt, a carbohydrate, a water-soluble amino acid or a combination thereof. In some embodiments, the first osmogen comprises magnesium chloride, magnesium sulfate, lithium chloride, sodium chloride, sodium sulfate, sodium phosphate, potassium chloride, potassium phosphate, sodium acetate, potassium acetate, magnesium succinate, sodium benzoate, sodium citrate, sodium ascorbate, sodium carboxymethyl cellulose, sucrose, sorbitol, mannitol, glucose, lactose, fructose, glycine, leucine, alanine, methionine, urea or a combination thereof. In some embodiments, some materials can serve as both of the first polymer and the first osmogen. Therefore, the barrier delaying the release of the first active ingredient (caused by the first polymer) and the diffusion of the liquid in the environment (caused by the first osmogen) can be adjusted for controlling the release of the first active ingredient by the selection of the suitable materials served as the first polymer and the first osmogen simultaneously.

In some embodiments, a weight ratio of the first active ingredient and the first polymer is from 1:1.5 to 1:7, such as 1:1.5, 1:2, 1:3, 1:4, 1:5, 1:6, 1:7 or any value in any abovementioned interval. In some embodiments, a weight ratio of the first active ingredient and the first osmogen is from 1:0.6 to 1:5, such as 1:0.6, 1:1, 1:1.5, 1:2, 1:3, 1:4, 1:5 or any value in any abovementioned interval. If the weight ratio is too low, the released percentage of the first active ingredient is reduced. If the weight ratio is too high, the released content of the first active ingredient is increased and the risk of the fluctuation of the first active ingredient plasma concentration is increased.

In some embodiments, step S110 further comprises mixing a first polymer, a first osmogen and a first active ingredient with an adhesive, a lubricant (such as magnesium stearate), a preservative, a filler, an acidifier, a colorant or a combination thereof. For example, HPMC (hypromellose) can be served as the adhesive to help the adhesion of the first polymer, the first osmogen and the first active ingredient. In some embodiments, the acidifier provides an acid microenvironment to help the dissolution of the first active ingredient. The acidifier is for example comprising citric acid, succinic acid, tartaric acid or a combination thereof.

In some embodiments, magnesium stearate can be last added for achieving the better lubricating efficiency.

In some embodiments, the method 100 further comprises mixing a second polymer and a second osmogen to form a push compartment mixture. Since the candidate materials of the second polymer and the second osmogen are respectively similar to which of the first polymer and the first osmogen, they are not repeated herein.

Now, refer to step S120, compressing and tableting the drug compartment mixture to form a core.

In some embodiments, the step S120 comprises compressing the drug compartment mixture to form a drug compartment; mixing the push compartment mixture with the drug compartment to form a core mixture; and compressing and tableting the core mixture to form the core. It should be emphasizing that mixing the push compartment mixture with the drug compartment after compressing the drug compartment mixture can provide the core with two layers which the drug compartment is stacked with the push compartment. Therefore, while the sustained release osmotic-controlled pharmaceutical composition absorbs liquid in the environment, the push compartment can expand to push the first active ingredient in drug compartment out of the sustained release osmotic-controlled pharmaceutical composition from an orifice in a portion of the semi-permeable membrane adjacent to the drug compartment, increasing the release content of the first active ingredient. In one embodiment, compared with xanthan gum, the selection of poly(ethylene oxide) for serving as the second polymer in the push compartment provides higher push force, achieving higher release of the first active ingredient (i.e., higher plateau in dissolution assay).

In some embodiments, before step S120, the method 100 further comprises granulating the drug compartment mixture, such as adding an organic solvent (such as ethanol) to the drug compartment mixture, sieving the drug compartment mixture to obtain drug compartment particles and drying the drug compartment particles. For example, drug compartment particles are dried at 40° C. to 60° C. for 20 mins to 40 mins. Through the procedure of granulating the drug compartment mixture, the particle size of drug compartment particles can be more uniform and are well distributed, thereby increasing uniformity and fluidity and increasing the release stability of the first active ingredients. In some embodiments, before mixing the push compartment mixture with the drug compartment, the method 100 further includes granulating the push compartment mixture with the procedures similar to which of granulating the drug compartment mixture for further increasing the fluidity of the push compartment mixture and stabilizing the weight of the core mixture in the following compressing and tableting step.

It's noted that the release percentages of the first active ingredient can be adjusted by the weight percentages of the materials in the core, such as the first active ingredient, the first polymer, the first osmogen, the second polymer, and the second osmogen, or the like.

In some embodiments, a weight percentage of the first active ingredient is from 2%-30% based on 100% by weight of the core, such as 2%, 5%, 10%, 15%, 20%, 25%, 30%, or any value between any interval of the above-mentioned values. If the weight percentage of the first active ingredient is too low, the barrier provided by the polymer (such as the first polymer or a combination of the first polymer and the second polymer) is too high, and the release efficiency of the first active ingredient is limited. If the weight percentage of the first active ingredient is too high, the risk of the fluctuation of the first active ingredient plasma concentration is increased.

In some embodiments, a weight percentage of the first polymer is from 5% to 80% based on 100% by weight of the core, such as 5%, 10%, 20%, 30%, 40%, 50%, 60%, 70%, 80%, or any value between any interval of the above-mentioned values. Generally, if the weight percentage of the first polymer is too low, the barrier provided by the first polymer is not enough, and the risk of the fluctuation of the first active ingredient plasma concentration is increased. If the weight percentage of the first polymer is too high, the barrier provided by the first polymer is too high, and the release efficiency of the first active ingredient is limited. In some preferable embodiments, a weight percentage of the first polymer is from 10% to 80% based on 100% by weight of the core.

In some embodiments, a weight percentage of the first osmogen is from 10% to 55% based on 100% by weight of the core, such as 10%, 20%, 30%, 40%, 50%, 55%, or any value between any interval of the abovementioned values. Generally, if the weight percentage of the first osmogen is too low, the pressure provided by the first osmogen is not enough, and the release efficiency of the first active ingredient is limited. If the weight percentage of the first osmogen is too high, the pressure provided by the first osmogen is too high, and the risk of the fluctuation of the first active ingredient plasma concentration is increased.

In some embodiments, a weight percentage of the second polymer is from 15% to 55% based on 100% by weight of the core, such as 15%, 20%, 30%, 45%, 50%, 55%, or any value between any interval of the abovementioned values. In some embodiments, a weight percentage of the second osmogen is from 5% to 55% based on 100% by weight of the core, such as 5%, 10%, 15%, 20%, 25%, 30%, 35%, 40%, 45%, 50%, 55%, or any value between any interval of the abovementioned values. In some preferred embodiments, a weight percentage of the second osmogen is from 5% to 15% based on 100% by weight of the core. Since the push compartment expands and provides the force to the drug compartment after liquid in the environment diffuses into the sustained release osmotic-controlled pharmaceutical composition, if the weight percentages of the second polymer or the second osmogen is too low, the force provided by the push compartment is not enough, the release efficiency of the first active ingredient is limited. If the weight percentages of the second polymer or the second osmogen is too high, the force provided by the push compartment is too high, and the risk of the fluctuation of the first active ingredient plasma concentration is increased.

In some embodiments, a weight percentage of a combination of the first polymer and the second polymer is from 30% to 80% based on 100% by weight of the core, such as 30%, 40%, 50%, 60%, 70%, 80%, or any value between any intervals of the abovementioned values. Generally, if the weight percentage of the combination of the first polymer and the second polymer is too low, the barrier provided by the first polymer and the second polymer is not enough, and the risk of the fluctuation of the first active ingredient plasma concentration is increased. Additionally, if the weight percentage of the combination of the first polymer and the second polymer is too low, for meeting the requirement of desired dose of the active ingredient, the volume of the sustained release osmotic-controlled pharmaceutical composition may be too large to be administrated. If the weight percentage of the combination of the first polymer and the second polymer is too high, the barrier provided by the first polymer and the second polymer is too high, and the release efficiency of the first active ingredient is limited.

Refer to step S130, mixing a membrane body material and a porogen in a solvent to form a membrane liquid.

In some embodiments, the membrane body material mainly forms semi-permeable membrane, in which the membrane body material comprises cellulose acetate, ethyl cellulose or a combination thereof. In some embodiments, the porogen is used to form pores in the semi-permeable membrane to allow liquid in the environment to flow into the sustained release osmotic-controlled pharmaceutical composition. Porogen comprises hydroxypropylcellulose, hypromellose, glycerin, propylene glycol, polyethylene glycol, sucrose, mannitol, lactose, sodium chloride or a combination thereof. In some embodiments, the solvent comprises a dual solvent system for dissolving the membrane body material and the porogen. For example, the dual solvent system is a combination of a water-phase solvent (such as water) and an organic solvent (such as acetone).

In some embodiments, the step S130 comprises mixing the membrane body material, the porogen and a plasticizer in a solvent, in which the plasticizer is used for increasing the strength of the semi-permeable membrane. In some embodiments, the plasticizer comprises triethyl citrate, propylene glycol, polysorbate 80, polyethylene glycol, polyoxymethylene, polyethylene oxide, sorbitan ester, triacetyl glyceride, diethyl phthalate, mineral oil, three sebacic acid, glycerin or a combination thereof.

In some embodiments, a weight percentage of the membrane body material and the porogen in the membrane liquid is from 2.5% to 7.5%, such as 2.5%, 5%, 7.5% or any value between any interval of the abovementioned values. If the weight percentage is too low, the time required for the following steps (spraying and drying) is elongated since the following steps should be performed for more times. If the weight percentage is too high, it's hardly to control the appropriate amount of the membrane body material and the porogen for coating on the core in the following steps.

Refer to step S140, spraying the membrane liquid on the core.

Refer to step S150, drying the membrane liquid to form a semi-permeable membrane coated on the core, in which the pores formed by the porogen and distributed in the membrane body, allowing the liquid in the environment to diffuse into the drug compartment and the push compartment.

In some embodiment, membrane liquid is further dried at a temperature of from 40° C. to 50° C. (such as 40° C., 45° C., 50° C. or any value between any interval of the abovementioned values) for 40 hrs to 60 hrs (such as 40 hrs, 45 hrs, 50 hrs, 55 hrs, 60 hrs or any value between any interval of the abovementioned values) after the step S150. If the temperature is too low or the time is too short, the residual solvent would be left out of the sustained release osmotic-controlled pharmaceutical composition. If the temperature is too high or the time is too long, the state of the first polymer or the second polymer may be changed.

In some embodiments, a weight percentage of the semi-permeable membrane is from 1% to 45% based on 100% by weight of the core, such as 1%, 5%, 10%, 15%, 20%, 25%, 30%, 35%, 40%, 45%, or any value between any interval of the abovementioned values. In some preferred embodiments, the weight percentage of the semi-permeable membrane is from 1% to 40% based on 100% by weight of the core. If the weight percentage is too low, the dissolution rate of the first ingredient is too high (i.e., the slope of the zero order release is too high), and the risk of the fluctuation of the first active ingredient is increased. If the weight percentage is too high, the dissolution rate of the first ingredient is too low (i.e., the slope of the zero order release is too low), and the release efficiency is not enough.

According to step S110 to step S150, the sustained release osmotic-controlled pharmaceutical composition comprising the core and the semi-permeable membrane coated on the core is provided, in which the core may only comprise the drug compartment or comprise the drug compartment and the push compartment depending on the clinical demand.

In some embodiments, when assayed in a Mcilvaine buffer in pH 3.8, a first dissolution percentage of the first active ingredient is from 0% to 30% within 2 hrs, such as 0%, 5%, 10%, 15%, 20%, 25%, 30% or any value between any interval of the abovementioned values. In some embodiments, when assayed in a Mcilvaine buffer in pH 3.8, a second dissolution percentage of the first active ingredient is from 30% to 100% within 10 hrs, such as 30%, 40%, 50%, 60%, 70%, 80%, 90%, 100% or any value between any interval of the above-mentioned values. It's noted that the dissolution rate of the first active ingredient can be kept at zero-order release rate for at least 4 hrs, so that the fluctuation of the first active ingredient plasma concentration can be avoided.

In some other embodiments, for keeping the supply of the first active ingredient in early release state (such as release state within 4 hrs), the method 100 further comprises mixing a film coating material in a aqueous solution to form a film coating liquid and spraying the film coating liquid on the semi-permeable membrane to form a film coating coated on the semi-permeable membrane. In some embodiments, the method 100 further comprises drying the film coating liquid after spraying the film coating liquid on the semi-permeable membrane similar to the step S150.

In some embodiments, the film coating material comprises a second active ingredient and a film material, in which the second active ingredient is similar to the first active ingredient. In some embodiments, the film material comprises hypromellose.

In some embodiments, a weight percentage of the film coating is from 2% to 25% based on 100% by weight of the core and the semi-permeable membrane, such as 2%, 5%, 7.5%, 10%, 15%, 20%, 25% or any value between any interval of the abovementioned values. In some embodiments, a weight percentage of the second active ingredient is from 10% to 60% based on 100% by weight of the first active ingredient in the core, such as 10%, 15%, 20%, 25%, 30%, 40%, 50%, 60% or any value between any interval of the abovementioned values. If the weight percentage is too low, the released percentage of the active ingredient (the first active ingredient and the second active ingredient) is limited. If the weight percentage is too high, the risk of the fluctuation of the active ingredient plasma concentration is increased.

In some embodiments, a weight percentage of the second active ingredient and the film material in the film coating liquid is from 5% to 15%, such as 5%, 10%, 15% or any value between any interval of the above-mentioned values. If the weight percentage is too low, the time required for the following steps (spraying) is elongated since the following step should be performed for more times. If the weight percentage is too high, it's hardly to control the appropriate amount of the second active ingredient and the film material.

In some embodiments, the method 100 further comprises forming at least one orifice by an artificial work, a laser, a mechanical method or other suitable orifice-making method. In some embodiments, the orifice was formed in a portion of the semi-permeable membrane adjacent to the drug compartment for allowing the first active ingredient push out of the sustained release osmotic-controlled pharmaceutical composition.

In some embodiments, when the sustained release osmotic-controlled pharmaceutical composition with the film coating is assayed in a Mcilvaine buffer in pH 3.8, a dissolution percentage of the active ingredient (the first active ingredient and the second active ingredient) is from 10% to 40% within 1 hr, such as 10%, 15%, 20%, 25%, 30%, 35%, 40% or any value between any interval of the abovementioned values. The dissolution percentage of the active ingredients within 1 hr can be adjusted according to the weight percentage of the second active ingredient or a weight ratio of the second active ingredient to the film material.

Method for Treatment

In another aspect of the disclosure, a method of treating a mental disease is provided, comprising administering the abovementioned sustained release osmotic-controlled pharmaceutical composition to a subject suffered from the mental disease. In some embodiment, the mental disease comprises schizophrenia, bipolar disorder, autism, depression or a combination thereof.

In another aspect of the disclosure, use of the abovementioned sustained release osmotic-controlled pharmaceutical composition in the manufacture of a medicament of treating a mental disease is provided.

In some embodiments, the medicament is in the form of tablet, capsule or enteric formulation.

In some embodiments, the medicament comprises excipients, such as fillers, disintegrants, adhesives, surfactants, flavoring agents, colorants and lubricants.

It should be understood that the above-described embodiments and the following examples are given by way of illustration, not limitation. Various changes and modifications within the scope of the present invention will become apparent to those skilled in the art from the present description.

I. Method of Preparing Sustained Release Osmotic-Controlled Pharmaceutical Composition

For clarifying the suitable formulation ratio and materials of the sustained release osmotic-controlled pharmaceutical composition, Example 1 to Example 9 are provided, and the methods of preparing Example 1 to Example 9 are described below in sequence.

### Example 1

1. Granulation of Drug Compartment
(1) Mix the materials except magnesium stearate according to Table 1 uniformly.
(2) Add appropriate amount of alcohol for granulation, and then pass the wet granules through a 20-mesh sieve.
(3) Dry the materials in a fluidized bed at 50° C. for 30 minutes, then screen the dry granules with the 20-mesh sieve to form particles.
(4) Mix the particles obtained by abovementioned (3) with magnesium stearate uniformly to obtain drug compartment particles.

2. Granulation of Push Compartment
(1) Mix the materials except magnesium stearate according to Table 1 uniformly.
(2) Add appropriate amount of alcohol for granulation, and then pass the wet granules through a 20-mesh sieve.
(3) Dry the materials in a fluidized bed at 50° C. for 30 minutes, then screen the dry granules with the 20-mesh sieve to form particles.
(4) Mix the particles obtained by abovementioned (3) with magnesium stearate uniformly to obtain push compartment particles.

3. Compressing and Tableting
(1) Compress the drug compartment particles in shape using a tableting machine.
(2) Add the push compartment particles over the compressed drug compartment particles, and then press the compressed drug compartment particles and the push compartment particles with a tablet machine to form a core with two layers comprising a drug compartment and a push compartment, following by the procedure of coating a semi-permeable membrane.

4. Coating of Semi-Permeable Membrane
(1) Dissolve the materials in Table 2 in a dual solvent system of acetone and water to form a 5% (w/w) membrane liquid, in which the weight ratio of acetone to water was 95:5.
(2) Spray the membrane liquid on the core (with two layers) obtained by the abovementioned Point 3 and increase weight of the semi-permeable membrane according to Table 2, in which the prescription percentage of Table 2 was based on 100% by weight of the core.
(3) Dry the tablet coated by the semi-permeable membrane in an oven at 45° C. for 48 hrs.

5. Formation of Orifice through Semi-Permeable Membrane

A orifice through the semi-permeable membrane of the tablet obtained by the abovementioned Point 4 is formed using a laser, in which the orifice was formed in a portion of the semi-permeable membrane adjacent to the drug compartment.

TABLE 1
Prescription of Core of Example 1 (291.2 mg/tablet)

| | Composition | Percentage (%) |
|---|---|---|
| Drug Compartment | Lurasidone HCl | 15.11 |
| | POLYOX ™ WSR N80 | 28.85 |
| | Povidone K29-32 | 1.37 |
| | Sodium chloride | 1.37 |
| | Succinic acid | 3.02 |
| | Magnesium stearate | 0.28 |
| Push Compartment | POLYOX ™ WSR 303 | 38.04 |
| | Sodium chloride | 9.95 |
| | Povidone K29-32 | 1.49 |
| | Butylated | 0.02 |
| | hydroxytoluene (BHT) | |
| | Red ferric oxide | 0.25 |
| | Magnesium stearate | 0.25 |

Note 1:
POLYOX ™ WSR N80 is poly(ethylene oxide with molecular weight of 2 × 10⁵ g/mole.

Note 2: POLYOX™ WSR 303 is poly(ethylene oxide with molecular weight of 7×10⁶ g/mole.

TABLE 2
Prescription of Semi-Permeable Membrane of Example 1

| | Composition | Percentage (%) |
|---|---|---|
| Semi-Permeable | Cellulose acetate 398 | 3.26 |
| Membrane | Polyethylene glycol 3350 | 0.17 |

### Example 2

The method of preparing Example 2 was basically according to Example 1, in which the prescription of the core was referred to Table 3, and the prescription of the semi-permeable membrane was referred to Table 4.

TABLE 3
Prescription of Core of Example 2 (394 mg/tablet)

| | Composition | Percentage (%) |
|---|---|---|
| Drug Compartment | Lurasidone HCl | 11.17 |
| | POLYOX ™ WSR N80 | 21.32 |
| | Povidone K29-32 | 1.02 |
| | Sodium chloride | 4.06 |
| | Succinic acid | 2.23 |
| | Magnesium stearate | 0.20 |
| Push Compartment | POLYOX ™ WSR 303 | 45.65 |
| | Sodium chloride | 11.93 |
| | Povidone K29-32 | 1.79 |
| | BHT | 0.03 |
| | Red ferric oxide | 0.30 |
| | Magnesium stearate | 0.30 |

TABLE 4
Prescription of Semi-Permeable Membrane of Example 2

| | Composition | Percentage (%) |
|---|---|---|
| Semi-Permeable | Cellulose acetate 398 | 32.00 |
| Membrane | Polyethylene glycol 3350 | 8.00 |

### Example 3

The method of preparing Example 3 was basically according to Example 1, in which the prescription of the core was referred to Table 5, and the prescription of the semi-permeable membrane was referred to Table 6.

In addition, Example 3 further comprised procedures to manufacture a film coating after the orifice was formed in the tablet. The film coating contained contains lurasidone or its pharmaceutical acceptable salt. Prescription of the film coating was referred to Table 7, in which the prescription percentage of Table 7 was based on 100% by weight of the tablet coated with the semi-permeable membrane.

1. Formation of Film Coating
(1) Prepare for a 10% (w/w) film coating liquid according to the prescription of Table 7, comprising dissolving hypromellose in water, following by adding lurasidone HCl and stirring the film coating liquid evenly.
(2) Sieve the film coating liquid through a 100-mesh sieve.
(3) Spray the film coating liquid on the tablet after forming the orifice by the laser, following by increasing weight of the film coating liquid according to Table 7.

TABLE 5
Prescription of Core of Example 3 (210.6 mg/tablet)

| | Composition | Percentage (%) |
|---|---|---|
| Drug Compartment | Lurasidone HCl | 11.87 |
| | POLYOX ™ WSR N80 | 10.68 |
| | Mannitol | 19.94 |
| | Sodium chloride | 2.37 |
| | Citric acid 1-hydrate | 4.75 |
| | Magnesium stearate | 0.38 |
| Push Compartment | POLYOX ™ WSR 303 | 19.23 |
| | Sodium chloride | 9.79 |
| | Mannitol | 20.49 |
| | Red ferric oxide | 0.25 |
| | Magnesium stearate | 0.25 |

TABLE 6
Prescription of Semi-Permeable Membrane of Example 3

| | Composition | Percentage (%) |
|---|---|---|
| Semi-Permeable | Cellulose acetate 398 | 6.77 |
| Membrane | Polyethylene glycol 3350 | 0.36 |

TABLE 7
Prescription of Film Coating of Example 3

| | Composition | Percentage (%) |
|---|---|---|
| Film Coating | Lurasidone HCl | 6.65 |
| | Hypromellose (5cP) | 13.30 |

### Example 4

The method of preparing Example 4 was basically according to Example 1, in which the prescription of the core was referred to Table 8, and the prescription of the semi-permeable membrane was referred to Table 9.

TABLE 8
Prescription of Core of Example 4 (360 mg/tablet)

| | Composition | Percentage (%) |
|---|---|---|
| Drug Compartment | Lurasidone HCl | 11.11 |
| | POLYOX ™ WSR N10 | 31.94 |
| | Hypromellose (6 cP) | 1.39 |
| | Sodium chloride | 1.39 |
| | Citric acid 1-hydrate | 2.22 |
| | Pregelatinized starch | 1.67 |
| | (fully pregelatinized) | |
| | Magnesium stearate | 0.28 |
| Push Compartment | POLYOX ™ WSR 303 | 38.04 |
| | Sodium chloride | 9.95 |
| | Povidone K29-32 | 1.49 |
| | BHT | 0.02 |
| | Red ferric oxide | 0.25 |
| | Magnesium stearate | 0.25 |

TABLE 9
Prescription of Semi-Permeable Membrane of Example 4

| | Composition | Percentage (%) |
|---|---|---|
| Semi-Permeable | Cellulose acetate 398 | 5.28 |
| Membrane | Polyethylene glycol 3350 | 0.28 |

### Example 5

The method of preparing Example 5 was basically according to Example 1, in which the prescription of the core was referred to Table 10, and the prescription of the semi-permeable membrane was referred to Table 11.

TABLE 10
Prescription of Core of Example 5 (291.2 mg/tablet)

| | Composition | Percentage (%) |
|---|---|---|
| Drug Compartment | Lurasidone HCl | 15.11 |
| | POLYOX ™ WSR N80 | 28.85 |
| | Povidone K29-32 | 1.37 |
| | Sodium chloride | 1.37 |
| | Succinic acid | 3.02 |
| | Magnesium stearate | 0.28 |
| Push Compartment | Xanthan gum | 38.05 |
| | Sodium chloride | 9.96 |
| | Povidone K29-32 | 1.51 |
| | Red ferric oxide | 0.24 |
| | Magnesium stearate | 0.24 |

TABLE 11
Prescription of Semi-Permeable Membrane of Example 5

| | Composition | Percentage (%) |
|---|---|---|
| Semi-Permeable | Cellulose acetate 398 | 6.52 |
| Membrane | Polyethylene glycol 3350 | 0.34 |

### Example 6

The method of preparing Example 6 was basically according to Example 1, in which the prescription of the core was referred to Table 12, and the prescription of the semi-permeable membrane was referred to Table 13.

TABLE 12
Prescription of Core of Example 6 (281.6 mg/tablet)

| | Composition | Percentage (%) |
|---|---|---|
| Drug Compartment | Lurasidone HCl | 14.20 |
| | POLYOX ™ WSR N10 | 29.83 |
| | Povidone K29-32 | 1.42 |
| | Sodium chloride | 1.42 |
| | Succinic acid | 2.84 |
| | Magnesium stearate | 0.28 |
| Push Compartment | POLYOX ™ WSR 303 | 38.04 |
| | Sodium chloride | 9.95 |
| | Povidone K29-32 | 1.49 |
| | BHT | 0.03 |
| | Red ferric oxide | 0.25 |
| | Magnesium stearate | 0.25 |

TABLE 13
Prescription of Semi-Permeable Membrane of Example 6

| | Composition | Percentage (%) |
|---|---|---|
| Semi-Permeable | Cellulose acetate 398 | 6.75 |
| Membrane | Polyethylene glycol 3350 | 0.36 |

### Example 7

The method of preparing Example 7 was described below, in which the prescription of the core was referred to Table 14, and the prescription of the semi-permeable membrane was referred to Table 15.

1. Granulation of Core
(1) Mix the materials except magnesium stearate according to Table 14 uniformly.
(2) Add appropriate amount of alcohol for granulation, and then pass the wet granules through a 20-mesh sieve.
(3) Dry the materials in a fluidized bed at 50° C. for 30 minutes, then screen the dry granules with the 20-mesh sieve to form particles.
(4) Mix the particles obtained by abovementioned (3) with magnesium stearate uniformly to obtain particles.

2. Compressing and Tableting

Compress the particles obtained by abovementioned Point 1 in shape using a tableting machine, following by the procedure of coating a semi-permeable membrane.

3. Coating of Semi-Permeable Membrane
(1) Dissolve the materials in Table 15 in a dual solvent system of acetone and water to form a 5% (w/w) membrane liquid, in which the weight ratio of acetone to water was 95:5.
(2) Spray the membrane liquid on the core obtained by the abovementioned Point 2 and increase weight of the semi-permeable membrane according to Table 15, in which the prescription percentage of Table 15 was based on 100% by weight of the core.
(3) Dry the tablet coated by the semi-permeable membrane in an oven at 45° C. for 48 hrs.

4. Formation of Orifice through Semi-Permeable Membrane

Form a orifice through the semi-permeable membrane of the tablet obtained by the abovementioned Point 3 using a laser.

TABLE 14
Prescription of Core of Example 7 (200 mg/tablet)

| | Composition | Percentage (%) |
|---|---|---|
| Core | Lurasidone HCl | 22.00 |
| (Drug Compartment) | POLYOX ™ WSR N80 | 48.40 |
| | Povidone K29-32 | 4.00 |
| | Sodium chloride | 16.00 |
| | Succinic acid | 8.80 |
| | Magnesium stearate | 0.80 |

TABLE 15
Prescription of Semi-Permeable Membrane of Example 7

| | Composition | Percentage (%) |
|---|---|---|
| Semi-Permeable | Cellulose acetate 398 | 6.65 |
| Membrane | Polyethylene glycol 3350 | 0.35 |

### Example 8

The method of preparing Example 8 was basically according to Example 3, in which the prescription of the core was referred to Table 16, the prescription of the semi-permeable membrane was referred to Table 17, and the prescription of the film coating was referred to Table 18.

TABLE 16
Prescription of Core of Example 8 (304.5 mg/tablet)

| | Composition | Percentage (%) |
|---|---|---|
| Drug Compartment | Lurasidone HCl | 11.17 |
| | POLYOX ™ WSR N80 | 21.32 |
| | Povidone K29-32 | 1.01 |
| | Sodium chloride | 4.07 |
| | Succinic acid | 2.23 |
| | Magnesium stearate | 0.20 |
| Push Compartment | POLYOX ™ WSR 303 | 45.65 |
| | Sodium chloride | 11.93 |
| | Povidone K29-32 | 1.79 |
| | BHT | 0.03 |
| | Red ferric oxide | 0.30 |
| | Magnesium stearate | 0.30 |

TABLE 17
Prescription of Semi-Permeable Membrane of Example 8

| | Composition | Percentage (%) |
|---|---|---|
| Semi-Permeable | Cellulose acetate 398 | 6.24 |
| Membrane | Polyethylene glycol 3350 | 0.33 |

TABLE 18
Prescription of Film Coating of Example 8

| | Composition | Percentage (%) |
|---|---|---|
| Film Coating | Lurasidone HCl | 3.08 |
| | Hypromellose (5cP) | 6.16 |

### Example 9

The method of preparing Example 9 was basically according to Example 1, in which the prescription of the core was referred to Table 19, and the prescription of the semi-permeable membrane was referred to Table 20.

TABLE 19
Prescription of Core of Example 9 (453.10 mg/tablet)

| | Composition | Percentage (%) |
|---|---|---|
| Drug Compartment | Lurasidone HCl | 11.17 |
| | POLYOX ™ WSR N80 | 21.32 |
| | Povidone K30 | 1.02 |
| | Sodium chloride | 4.06 |
| | Succinic acid | 2.23 |
| | Magnesium stearate | 0.20 |
| Push Compartment | POLYOX ™ WSR 303 | 45.65 |
| | Sodium chloride | 11.93 |
| | Povidone K30 | 1.79 |
| | BHT | 0.03 |
| | Red ferric oxide | 0.30 |
| | Magnesium stearate | 0.30 |

TABLE 20
Prescription of Semi-Permeable Membrane of Example 9

| | Composition | Percentage (%) |
|---|---|---|
| Semi-Permeable | Ethocel 100 FP | 9.00 |
| Membrane | HPMC E5LV | 1.1 |
| | Triethyl Citrate | 1.1 |
| | Polyethylene glycol 3350 | 3.9 |

Note 3: Ethocel 100 FP is a kind of ethylcellulose.

Note 4: HPMC E5LV is a kind of hypromellose.

II. Dissolution Assay

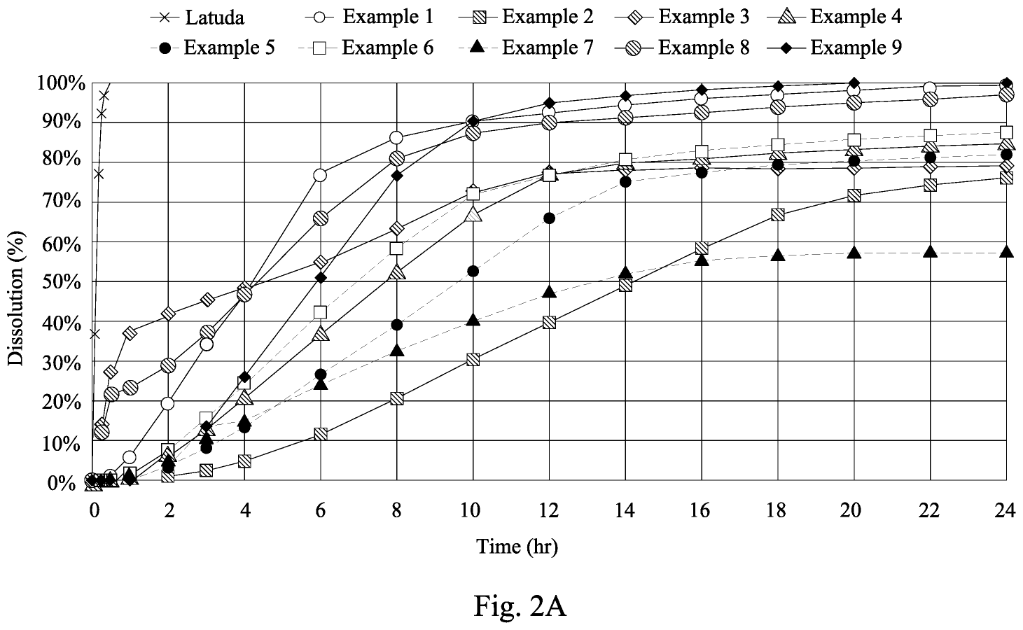

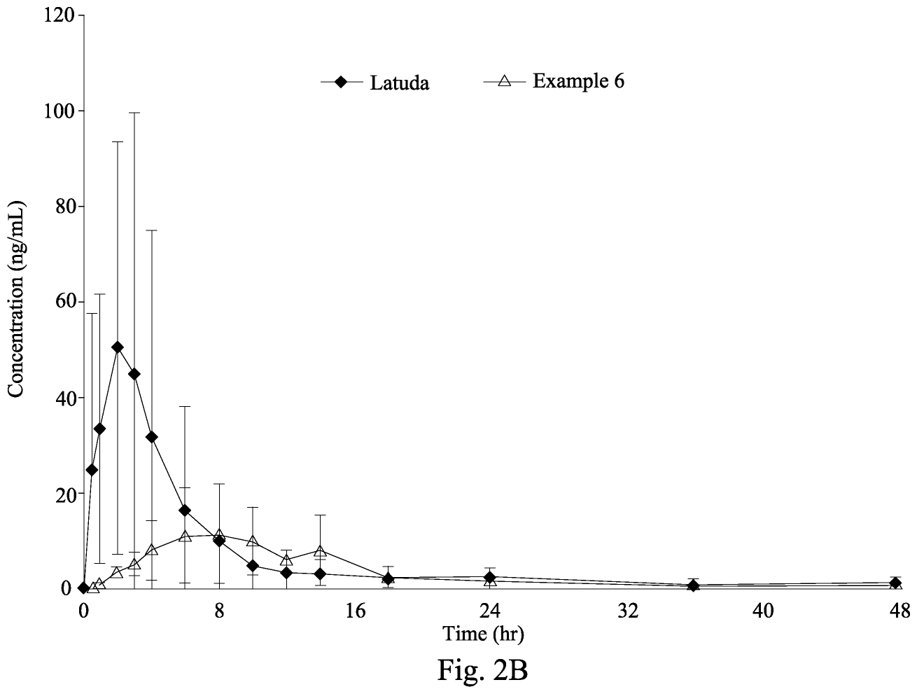

The dissolution assay for comparing Example 1 to Example 9 and the commercial product (Latuda®) was conducted in 900 mL of Mcilvaine buffer (0.025 M Citric acid Solution+0.05M Na₂HPO₄) at pH 3.8 using the paddle method with the rotation speed of 50 rpm, and the time points for sampling were 0, 1, 2, 3, 4, 6, 8, 10, 12, 14, 16, 18, 20, 22, and 24 hours. The result of the dissolution assay was shown in FIG. 2A.

FIG. 2A represented that compared with Latuda®, the release time of the active ingredient (lurasidone HCl) was prolonged in Examples 1 to 9. Specifically, compared with Latuda®, which represented 100% release of the active ingredient within only 1 hr, the dissolution percentages of the active ingredient of Example 1 to Example 9 were from 0% to 50% within 2 hrs and from 30% to 100% within 10 hrs.

Furthermore, comparing to the sustained release osmotic-controlled pharmaceutical composition without the push compartment (Example 7), it was observed that the sustained release osmotic-controlled pharmaceutical composition with the push compartment (Examples 1-6 and 8-9) performed the higher plateau. That is, the push compartment increases the release content of the active ingredient.

Moreover, comparing to the sustained release osmotic-controlled pharmaceutical composition without the film coating (Examples 1-2, 4-7 and 9), it was observed that the sustained release osmotic-controlled pharmaceutical composition coated with the film coating (Example 3 and Example 8) performed the increased release of the active ingredient within 2 hrs.

Furthermore, refer to Example 1 and Example 5, differing from one material of the push compartment (Example 1: poly(ethylene oxide; Example 5: xanthan gum) and the weight percentage of the semi-permeable membrane (Example 1: 3.43%; Example 5: 6.86%). Compared with Example 1, it was observed in Example 5 that the higher weight percentage of the semi-permeable membrane performed the reduced release slope (lower release rate), and the selection of xanthan gum, instead of poly(ethylene oxide) in Example 1, performed the lowered plateau.

Refer to Example 2 and Example 8, differing from the prescription of the semi-permeable membrane (such as, Example 2 was 40%; Example 8 was 6.57%) and whether the filming coating was coated (Example 2: no filming coating; Example 8: filming coating). Compared with Example 2, it was observed in Example 8 that the lower weight percentage of the semi-permeable membrane performed the higher release slope (higher release rate), and the filming coating increased the early release of the active ingredient.

Refer to Example 8 and Example 9, mainly differing from the prescription of the semi-permeable membrane (such as, the membrane body of Example 8 was cellulose acetate, but which of Example was ethyl cellulose (Ethocel 100 FP), and the weight ratio of the membrane body to the porogen of Example 8 was higher than which of Example 9) and whether the filming coating was coated (Example 8: filming coating; Example 9: no filming coating). Compared with Example 9, it was observed in Example 8 that the selection of cellulose acetate for serving as the membrane body, instead of ethyl cellulose in Example 9, and the higher weight ratio of the membrane body to the porogen performed the reduced release slope (lower release rate), and the filming coating increased the intermediate release of the active ingredient.

According to FIG. 2A, since the dissolution profile of Example 6 efficiently delayed the release of lurasidone and presented the profile preferably fitting the desirable dose, Example 6 was selected to conduct the following animal study.

III. Animal Study

The diffusions of the sustained release osmotic-controlled pharmaceutical composition manufactured by Example 6 and Latuda® were studied in beagle dogs with a single-dose parallel test, in which the beagle dogs were divided into two groups. Within 20 to 30 minutes after the meal, each group was given Example 6 or a tablet of 40 mg of Latuda®, and the time points for sampling were 0, 0.5, 1, 2, 3, 4, 6, 8, 10, 12, 14, 18, 24, 36 and 48 hours. The blood concentrations of lurasidone HCl of two groups were shown in FIG. 2B.

It was represented in FIG. 2B that compared with Latuda®, Cmax of the sustained release osmotic-controlled pharmaceutical composition of the present disclosure (such as Example 6) was obviously is reduced, and the concentration curve was relatively flat without obvious peaks, indicating that the sustained release osmotic-controlled pharmaceutical composition performed the effect of stabilizing the drug concentration in the blood.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A sustained release osmotic-controlled pharmaceutical composition, comprising:
a core, comprising a drug compartment, wherein the drug compartment comprises a first active ingredient, a first polymer and a first osmogen, and the first active ingredient comprises lurasidone, a pharmaceutical acceptable salt of the lurasidone or a combination thereof; and
a semi-permeable membrane coated on the core, wherein the semi-permeable membrane comprises a membrane body and at least one pore distributed in the membrane body.

2. The sustained release osmotic-controlled pharmaceutical composition of claim 1, wherein a weight percentage of the first active ingredient is from 2%-30% based on 100% by weight of the core.

3. The sustained release osmotic-controlled pharmaceutical composition of claim 1, wherein a weight percentage of the first polymer is from 5% to 80% based on 100% by weight of the core.

4. The sustained release osmotic-controlled pharmaceutical composition of claim 1, wherein the first polymer comprises poly(methyl methacrylate), microcrystalline cellulose, methylcellulose, hydroxyethyl cellulose, hydroxypropyl cellulose, poly(ethylene oxide), polyoxypropylene, polyvinylpyrrolidone, carbomer, sodium carboxymethyl starch, carboxymethyl cellulose, a sodium salt of the carboxymethyl cellulose, cross-linked sodium carboxymethyl cellulose, xanthan gum or a combination thereof.

5. The sustained release osmotic-controlled pharmaceutical composition of claim 1, wherein the first osmogen comprises a water-soluble salt, a carbohydrate, a water-soluble amino acid or a combination thereof, and a weight percentage of the first osmogen is from 10% to 55% based on 100% by weight of the core.

6. The sustained release osmotic-controlled pharmaceutical composition of claim 1, wherein the first osmogen comprises magnesium chloride, magnesium sulfate, lithium chloride, sodium chloride, sodium sulfate, sodium phosphate, potassium chloride, potassium phosphate, sodium acetate, potassium acetate, magnesium succinate, sodium benzoate, sodium citrate, sodium ascorbate, sodium carboxymethyl cellulose, hypromellose, sucrose, sorbitol, mannitol, glucose, lactose, fructose, glycine, leucine, alanine, methionine, urea or a combination thereof.

7. The sustained release osmotic-controlled pharmaceutical composition of claim 1, wherein the drug compartment further comprises an acidifier.

8. The sustained release osmotic-controlled pharmaceutical composition of claim 1, wherein a weight percentage of the semi-permeable membrane is from 1% to 45% based on 100% by weight of the core.

9. The sustained release osmotic-controlled pharmaceutical composition of claim 1, wherein the membrane body comprises cellulose acetate, ethyl cellulose or a combination thereof.

10. The sustained release osmotic-controlled pharmaceutical composition of claim 1, wherein the core further comprises a push compartment, and the push compartment comprises a second polymer and a second osmogen.

11. The sustained release osmotic-controlled pharmaceutical composition of claim 10, wherein a weight percentage of the second polymer is from 15% to 55% based on 100% by weight of the core, and a weight percentage of the second osmogen is from 5% to 55% based on 100% by weight of the core.

12. The sustained release osmotic-controlled pharmaceutical composition of claim 1, wherein when assayed in a Mcilvaine buffer in pH 3.8, a first dissolution percentage of the first active ingredient is from 0% to 30% within 2 hrs, and a second dissolution percentage of the first active ingredient is from 30% to 100% within 10 hrs.

13. The sustained release osmotic-controlled pharmaceutical composition of claim 1, further comprising a film coating coated on the semi-permeable membrane.

14. The sustained release osmotic-controlled pharmaceutical composition of claim 13, wherein a weight percentage of the film coating is from 2% to 25% based on 100% by weight of the core and the semi-permeable membrane.

15. The sustained release osmotic-controlled pharmaceutical composition of claim 13, wherein the film coating comprises a second active ingredient and the second active ingredient comprises lurasidone, a pharmaceutical acceptable salt of the lurasidone or a combination thereof, wherein a weight percentage of the second active ingredient is from 10% to 60% based on 100% by weight of the first active ingredient in the core.

16. The sustained release osmotic-controlled pharmaceutical composition of claim 15, wherein when assayed in a Mcilvaine buffer in pH 3.8, a dissolution percentage of the first active ingredient and the second active ingredient is from 10% to 40% within 1 hr.

17. A method of preparing a sustained release osmotic-controlled pharmaceutical composition, comprising:
mixing a first active ingredient, a first polymer and a first osmogen to form a drug compartment mixture, wherein the first active ingredient comprises lurasidone, a pharmaceutical acceptable salt of the lurasidone or a combination thereof;
compressing and tableting the drug compartment mixture to form a core;
mixing a membrane body material and a porogen in a solvent to form a membrane liquid;
spraying the membrane liquid on the core; and
drying the membrane liquid to form a semi-permeable membrane coated on the core.

18. The method of claim 17, further comprising:
mixing a second polymer and a second osmogen to form a push compartment mixture; and
wherein compressing and tableting the drug compartment mixture comprises:
compressing the drug compartment mixture to form a drug compartment;
mixing the push compartment mixture with the drug compartment to form a core mixture; and
compressing and tableting the core mixture to form the core.

19. The method of claim 17, further comprising:
mixing a film coating material in an aqueous solution to form a film coating liquid; and
spraying the film coating liquid on the semi-permeable membrane to form a film coating coated on the semi-permeable membrane.

20. A method of treating a mental disease, comprising administering the sustained release osmotic-controlled pharmaceutical composition of claim 1 to a subject suffered from the mental disease.
